(19) United States
(12) Patent Application Publication
(10) Pub. No.: US 2024/0066190 A1
(43) Pub. Date: Feb. 29, 2024

(54) SYSTEM AND METHOD FOR PREVENTING DONOR REACTIONS DURING BLOOD DONATION

(71) Applicant: Fenwal, Inc., Lake Zurich, IL (US)

(72) Inventors: Thomas Gniadek, Deerfield, IL (US); Kyungyoon Min, Kildeer, IL (US)

(21) Appl. No.: 18/457,870

(22) Filed: Aug. 29, 2023

Related U.S. Application Data
(60) Provisional application No. 63/402,576, filed on Aug. 31, 2022.

(51) Int. Cl. A61M 1/02

(52) U.S. Cl. CPC A61M 1/024; A61M 2205/502; A61M 2230/005; A61M 2230/04; A61M 2230/50

(57) ABSTRACT

A method and system for donating blood and/or blood components is disclosed. The method and system include at least one adjustment mechanism to alter at least one feature of a blood draw in order to minimize and/or prevent donor reactions.

### CROSS-REFERENCE TO RELATED APPLICATIONS

This application claims the benefit of and priority of U.S. Provisional Patent Application Ser. No. 63/402,576, filed Aug. 31, 2022, the contents of which are incorporated by reference herein.

### FIELD OF THE INVENTION

The present disclosure is directed to a blood donation system and a method for anticipating and preventing donor reactions. The system monitors donor physiological parameters and makes adjustments to the system accordingly.

### BACKGROUND

Blood may be drawn from the arm or hand of a patient or donor for any of a number of reasons. Whole blood may be collected and donated to hospitals or other patient care centers. Alternatively, whole blood may be collected and separated into its components and the components may also be collected, while returning other components to the donor. The process of withdrawing blood, collecting some blood components and returning other components is commonly referred to as apheresis. During apheresis procedures, for example, blood is drawn from a patient or donor, a specific blood component is separated from the whole blood and removed and/or collected, and the remaining blood components are returned to the patient or donor, all in a continuous flow mode.

Apheresis and other blood draw procedures can range from approximately 30 minutes to 10 hours. During a blood donation procedure such as, but not limited to apheresis, donors can experience reactions caused by psychological factors or sudden intravascular depletion. Specifically, donors can experience vasovagal reactions or vasoconstriction that can cause sweating, nausea, dizziness, and/or fainting. During an apheresis procedure, citrate toxicity can also occur if the amount of citrate (which is typically present in the anticoagulant used in blood collection and processing) returned to the donor is too high for the donor's system to process effectively. Currently, reaction management or prevention strategies are implemented on a donor-specific basis by the phlebotomist, which therefore rely on the donor reporting early symptoms of a reaction and the phlebotomist taking time to act on those. Notwithstanding the best efforts of phlebotomists or blood center operator to address donor reactions, they remain common occurrences and can at times be significant.

Studies have shown that physiologic changes may occur prior to the onset of a full-blown donor reaction. Thus, it would be desirable to provide a system that monitors such physiologic changes before a full-blown reaction occurs, and adjusts certain conditions of the blood draw/apheresis procedure to prevent donor reactions.

### SUMMARY

There are several aspects of the present subject matter which may be embodied separately or together in the devices and systems described and claimed below. These aspects may be employed alone or in combination with other aspects of the subject matter described herein, and the description of these aspects together is not intended to preclude the use of these aspects separately or the claiming of such aspects separately or in different combinations as set forth in the claims appended hereto.

According to a first aspect, the present disclosure is directed to a method for reducing donor reactions during a blood donation using an automated blood draw device. The blood draw device includes a controller. The method includes initiating a blood draw, monitoring at least one donor parameter during the blood draw, and analyzing the at least one donor parameter. The method further includes predicting the probability of a reaction in a donor and adjusting at least one feature or condition of the blood draw so that a donor reaction can be minimized or prevented if a reaction is determined to be probable. The method (after initiating the blood draw) is repeated until the blood draw is complete.

In a second aspect, the present disclosure is directed to a system for performing a blood draw procedure. The system includes a vein access device and a fluid circuit comprising tubing connected to a vein access device and at least one blood collection container for collecting blood and/or blood components. The system also includes at least one donor parameter monitoring device, at least one adjusting mechanism for adjusting at least one feature of a blood draw, and a programmable user interface. The system further includes a controller configured to communicate with the donor parameter monitoring device, the at least one adjusting mechanism, and the user interface. The controller is configured to: initiate a blood draw, monitor at least one donor parameter during the blood draw, analyze at least one donor parameter, predict the probability of a reaction in a donor, and adjust at least one feature or condition of the blood draw so that a donor reaction can be minimized or prevented if a reaction is determined to be probable. The controller may repeat these steps until the blood draw is complete.

These and other aspects of the present subject matter are set forth in the following detailed description of the accompanying drawings.

### BRIEF DESCRIPTION OF THE DRAWINGS

FIG. 1 is a schematic illustration of a blood donation system of the type that may be advantageously used in the present disclosure.

FIG. 2 is a schematic representation of the possible components of a blood donation system of the type that may be advantageously used in the present disclosure.

FIG. 3 is a schematic representation of the possible components of a blood donation system of the type that may be advantageously used in the present disclosure.

### DETAILED DESCRIPTION

The embodiments disclosed herein are for the purpose of providing an exemplary description of the present subject matter. They are, however, only exemplary and not exclusive, and the present subject matter may be embodied in various forms. Therefore, specific details disclosed herein are not to be interpreted as limiting the subject matter as defined in the accompanying claims.

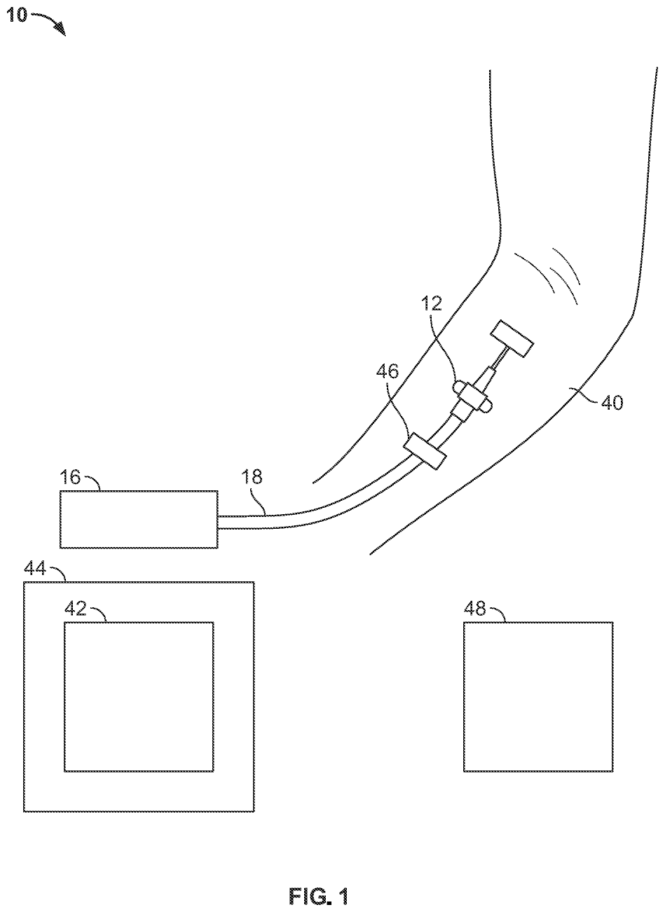

FIGS. 1-3 show components of an automated blood donation system that embody various aspects of the present subject matter. “Blood” includes without limitation whole blood and blood components, such as red cells, white cells, plasma, and platelets. By “automated,” it is meant that the apparatus can be programmed to carry out the blood donation without substantial operator involvement. Of course, even in the automated system of the present disclosure, it will be understood that some operator activity may be needed, including inserting the venous access device(s), the loading of the disposable fluid circuits, and entering processing parameters. Additional manual steps may be required as well. However, the apparatus can process blood through the disposable circuit(s) described below and make adjustments to the features of the blood draw (such as by an adjustment mechanism) without substantial operator intervention.

The blood donation system of the current disclosure will typically employ a disposable, single-use fluid processing assembly, module or circuit through which the fluid(s) flow, and a durable reusable hardware module to which the disposable is mounted or otherwise cooperatively engaged to control the flow of fluids through the disposable module and the donating of the blood or blood components.

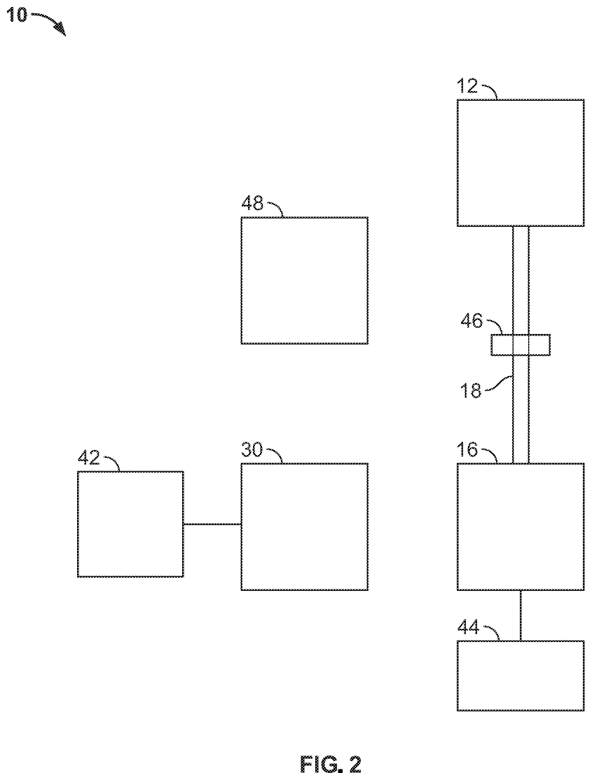

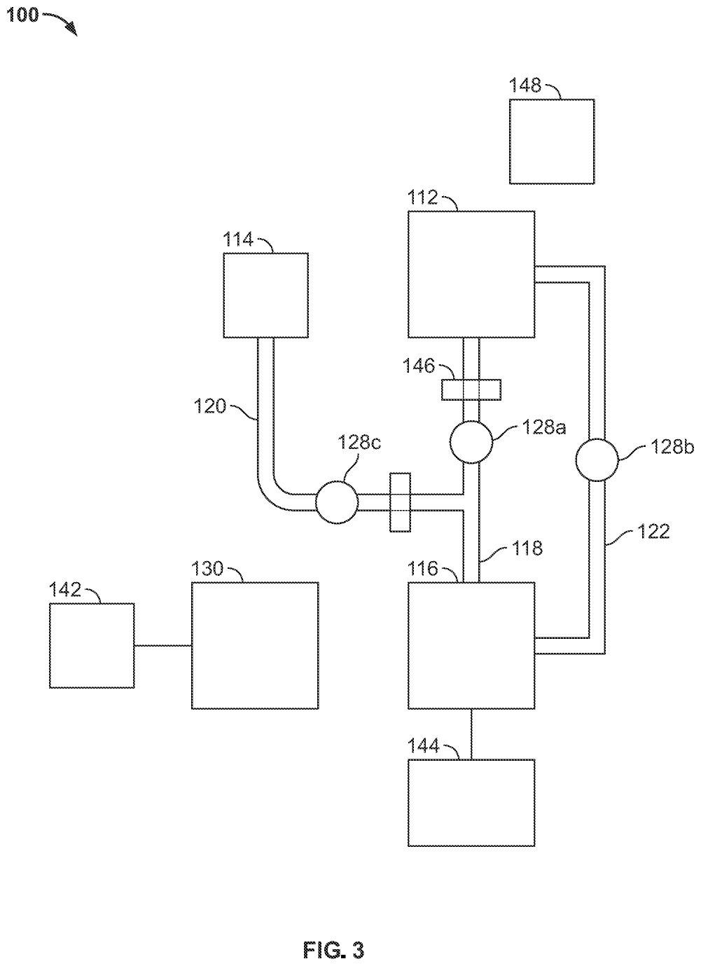

Depicted in the figures are two different schematics for different embodiments of the current blood donation system. FIGS. 1 and 2 depict systems for whole blood donation and FIG. 3 depicts a system for blood component donation (and return of remaining components to the donor), such as by apheresis. There may be overlap in components of the system and like numbers (for example, 12 and 112) indicate similar or identical components.

The donation system 10, as shown for illustration only, comprises a vein access, generally at 12, a blood storage container 16, a whole blood draw or flow path or line 18 for fluid flow from the vein access at the donor/patient 40 to the container. The system 10 further includes a controller 30 (FIG. 2) and input 42, which although illustrated together, may be separate, a patient parameter monitoring device 48, and at least one adjustment mechanism or mechanisms 44, 46 (several are illustrated in the figures). Fluid flow rates through the respective flow lines may be controlled by adjustment mechanism 44,46 under the command of a central processor or controller 30 which may receive input from the donor parameter monitoring device 48, all as described in more detail below.

The donation system 100, as shown in FIG. 3 for illustration only, comprises a vein access, generally at 112, an anticoagulant source 114, a blood processing container or assembly 116, a whole blood draw or flow path or line 118 for fluid flow from the vein access to the container or blood processing assembly 116, an anticoagulant fluid flow path or line 120 for fluid flow from the anticoagulant source to the whole blood draw line, and a return flow path or line 122 for returning one or more blood components from the processing assembly or container 116 to the donor or patient via the vein access 112. Fluid flow rates through the respective flow lines may be controlled by pumps 128a, 128b, and 128c on the whole blood, return, and anticoagulant flow paths or lines and optionally by adjustment mechanism 144,146 (if included), under the command of a central processor or controller 130 which may receive input from the donor parameter monitoring device 148, all as described in more detail below.

The above description of the systems 10 and 100 are intended to be illustrative and not exhaustive. For example, although various adjustment mechanisms are included in the figures, the system may only include one adjustment mechanism and the location can vary from as depicted in the figures. Further, the blood container is depicted as a single container, but it may include a plurality of containers and be a component of a blood separation system. Also, although a number of pumps are shown (or in the case of system 10, not shown) it is within the current disclosure to have a number of pumps present in either system or none at all. Other system components, such as a source of saline priming fluid, leukocyte reduction filters, sensors and the like, although not illustrated in the figures may be included in the system as desired.

Turning now to the various illustrated components, the vein access 12, 112 may include, for example, a single vascular access member such as a phlebotomy needle, vascular catheter or other access device for use in single access-site (sometimes called “single-needle”) procedures, in which whole blood is alternately drawn from a patient or donor and blood or blood components are alternately returned. The vein access also may include a pair of such access members for vascular access at different locations to permit simultaneous withdrawal of whole blood and return of blood or blood components.

The vein access 12, 112 is fluidly connected, such as by flexible plastic tubing, with a blood container 16, 116 via a whole blood draw or flow line 18, 118 as part of a fluid flow circuit. It may be noted here that the fluid flow lines of the system 10, 100 may have a variety of configurations and be made of any suitable material. For example, the flow lines may be provided as tubular conduits formed of either flexible tubing or the flow path may be preformed in a rigid plastic flow control cassette that is operated by solenoid, pneumatic or other valve arrangement to control flow direction through the cassette, as illustrated, for example in U.S. Pat. No. 5,538,405 to Patno et al. or U.S. Pat. No. 6,481,980 to Vandlik et al., both of which are incorporated by reference herein, or may be of any other suitable configuration.

The fluid flow circuit or flow set is intended to be a sterile, single use, disposable item. Before beginning a given blood donation procedure, the operator mounts the various components of the fluid flow circuit into the blood donation system. The controller implements the procedure based upon preset protocols, taking into account other input from the operator. Upon completing the procedure, the operator removes the fluid flow circuit from association with the blood donation system. The portions of the fluid flow circuit holding the collected blood component or components (e.g., collection containers or bags) are removed and retained for storage, transfusion, or further processing. The remainder of the fluid flow circuit is removed and discarded.

The blood container 16, 116 may be variously provided without departing from the scope of the present disclosure, and various devices may, for example, be used to obtain the treatment of blood and/or the separation of blood into its constituents, if desired. The blood container may simply be a bag or other type of holding container for storing donated blood. However, the blood container may also be connected to a separator or other blood processing components for separating different blood components. For example, the blood can be processed through a known centrifugal separation chamber, such as employed in the ALYX® or AMICUS® separators marketed by Fenwal, Inc. of Lake Zurich, Illinois, a subsidiary of Fresenius-Kabi of Bad Homburg, Germany, or centrifugal blood separators made by other manufacturers including Haemonetics Inc. of Braintree, Massachusetts; or CaridianBCT of Lakewood, Colorado. Static or moving membrane type separators may also be used to remove a particular blood component or constituent. More specifically, suitable blood processing assemblies, which the blood container 16, 116 are a component of, may include, but are not limited to, the centrifugal or spinning filtration membrane apheresis systems, such as those described in greater detail in U.S. Pat. No. 4,526,515 to DeVries; U.S. Pat. No. 5,194,145 to Schoendorfer; U.S. Pat. No. 6,312,607 to Brown et al.; U.S. Pat. No. 6,524,231 to Westberg et al.; U.S. Pat. No. 4,094,461 to Kellogg et al.; U.S. Pat. No. 7,052,606 to Gibbs et al.; U.S. Pat. No. 4,300,717 to Latham and U.S. Patent Application Publication No. 2009/0215602 to Min et al., all of which are hereby incorporated by reference.

The containers and the plastic tubing of the fluid circuit may be made of conventional medical grade plastic that can be sterilized by sterilization techniques commonly used in the medical field such as, but not limited to, radiation or autoclaving. Plastic materials useful in the manufacture of containers and of the tubing in the circuits disclosed herein include plasticized poly(vinyl chloride). Other useful materials include acrylics. In addition, certain polyolefins may also be used.

A variety of different disposable fluid flow circuits may be used in combination with the blood donation system, with the appropriate fluid flow circuit depending on the donation procedure to be carried out using the system.

The donation system 10, 100 may include one or more donor parameter monitoring devices to monitor donor parameters. Parameters monitored by the device(s) as part of the system may include, but are not limited to pulse, temperature, blood pressure, heart rate, sweat, facial expression, nerve conduction, and any other parameters for monitoring a person. The type of monitoring device(s) utilized in the system will depend on the donor parameters being monitored. For instance, a thermometer may be used for temperature monitoring. For blood pressure monitoring a cuff or other equivalent device may be used as a sensor. An infrared camera may be used to monitor temperature changes or heart rate. The monitoring device may also be a pulse oximeter, which can detect pulse change and/or oxygen levels in the blood. Additionally, the monitoring device may also be an electrocardiogram (EKG) which can monitor heart activity. The donor parameter monitoring devices may operate automatically and intermittently (such as every 2, 5, or 10 minutes) or continuously as programmed by the controller for monitoring donor parameters.

The system may utilize one or more types of adjustment mechanism during a whole blood or blood component donation to adjust at least one feature of the blood draw. The feature may include the height/position of a component of or of the fluid circuit and/or flow rate through one or more flow lines. In one embodiment, the adjustment mechanism is a device which moves or alters a portion of the blood donation system 10, 100. This portion, for example, can be blood container 16, 116.

The adjustment mechanism can be mechanical, pneumatic, hydraulic or ultrasonic. Any adjustment mechanism can be used that can move or adjust the blood container or flow line. The adjustment mechanism may also be a pump or clamp. By way of example, the adjustment mechanism may be electromechanically actuatable, utilizing electrical, pneumatic, or hydraulic fluid pressure energy. This can also be done ultrasonically. In one embodiment, the adjustment mechanism may comprise a screw, an axle and a connection member. The adjustment mechanism can be flexible or rigid and can be a chain, belt or cable or a rod or shaft. In yet another exemplary embodiment, the adjustment mechanism can comprise a cam and a motor which is driven in forward and reverse directions to rotate the cam in opposite directions, thereby moving the blood container in opposite directions. The adjustment mechanism may alternatively (or additionally) include a magnetic linear motor. In another embodiment, the adjustment mechanism may include a telescoping member.

The adjustment mechanism can alternatively move or adjust at least one flow line of the donation system. The donation system 10, 110 can include various clamps as adjustment mechanism configured to interact with and act on the flow lines of a fluid flow circuit on the system. As examples only, the tubing clamp or clamps may be solenoid pinch valves, motor-driven rotary pinch valves, linear actuators, stop cocks or any other type of automated clamping or valve device known in the art. The valves, in response to a command by the controller, may close or constrict partially to alter the flow rate on a flow line.

Additionally, the at least one adjustment mechanism may also include a pump or pumps along any of the flow lines of donation systems 10, 100. The system may also include a plurality of pumps to cause fluid to flow through the fluid flow circuit. The pumps may be differently or similarly configured and/or function similarly or differently from each other. The pump or pumps can regulate the flow of blood from the donor, the flow of blood components to the donor, and/or the anticoagulant that is added to the blood withdrawn. FIG. 3 shows a number of pumps associated with the various flow lines in system 100. The pumps 128a-128c are optional. Optionally a pump, specifically on the flow line 18, may be included in system 10. The pumps may be of any suitable construction or operate on any suitable principle. For example, one or more of the pumps may be a peristaltic pump, which operates by progressively compressing flow path tubing to move fluid therethrough. Peristaltic pumps are widely known in the apheresis and blood processing field. Alternatively, one or more of the pumps may be a diaphragm type pump that operates by alternately drawing fluid into and expelling it from a pump chamber. An example of such a pump chamber is found in the Fenwal ALYX® blood processing system, in which pump chambers are pre-formed into a rigid plastic cassette that also includes a flow path labyrinth. A flexible membrane overlies at least one side of the cassette and is operable under pneumatic-control, via the controller, with various portions of the membrane acting as a pump membrane in association with the pump chambers and other portions acting as flow control valves in association with the fluid flow paths. This arrangement is shown in greater detail in U.S. Pat. No. 6,524,231 to Westberg et al., incorporated by reference above. Other types of pumps may also be used that may be controlled by the controller 30, 130 to control flow through the system.

The donation system may include an anticoagulant pump, a whole blood pump, and/or a return pump. The anticoagulant pump 128c (if provided) may be adapted for continuously adding anticoagulant from the anticoagulant source 114 to the drawn whole blood in the system 100 at an anticoagulant flow rate AC. The whole blood pump 128a (if provided) may be adapted for drawing whole blood from a donor at a draw flow rate WB. The return pump 128b (if provided) may be adapted for returning processed blood (or a portion thereof) to a donor at a return flow rate R.

The donation system 10, 100 may also include weight scales for blood or blood component containers and may be provided with a plurality of hooks or supports that may support various components of the fluid flow circuit or other suitably sized and configured objects.

For controlling the donation procedures, including the relative fluid flow rates, the system 10, 100 further includes the controller 30, 130, which is configured to control the operation of the system 10,100. The controller 30, 130 may be provided as a computer or associated programmable microprocessor or other known mechanism for controlling one or more of the elements of the system 10, 100 in accordance with the procedure and steps set forth herein. Alternatively, a plurality of controllers may be employed rather than just a single controller 30, 130.

As is also illustrated throughout the Figures, the controller 30, 130 may be coupled to one or more of the structures described above, for example to receive information (e.g., in the form of signals) from these structures or to provide commands (e.g., in the form of signals) to these structures to control the operation of the structures. As illustrated, the controller 30, 130 may be coupled to at least one adjustment mechanism and at least one patient parameter sensor. Additionally, the controller 30, 130 may be coupled to pumps, and the drive or centrifuge separator (if included in the donation system) to provide commands to those devices to control their operation. The controller 30, 130 may be directly electrically connected to these structures to be coupled to them, or the controller 30, 130 may be directly connected to other intermediate equipment that is directly connected to these structures to be coupled to them.

The controller may be connected to at least one input. The at least one input 42, 142 may include a number of different devices according to the embodiments described herein. For example, the input 42, 142 could include a keyboard, keypad or touchscreen by which a user may provide information and/or instructions to the controller 30, 130. Alternatively, the input 42, 142 may be a touch screen, such as may be used in conjunction with a video display. The input could also include a reader or scanner, such as a barcode reader or scanner or an RFID reader. The assembly of the input/touch screen and video display may be one of the afore-mentioned structures to which the controller 30, 130 is coupled from which the controller 30, 130 receives information and to which the controller 30 provides commands. According to still other embodiments, the input 42, 142 may be in the form of computer equipment that permits the blood donation system including the controller 30, 130 to communicate (whether via wires, cables, etc. or wirelessly) with other systems over a local network, or with other cell processing systems or other computer equipment (e.g., a server) over local networks, wide area networks, or the Internet. According to such an embodiment, the input may include an internal transmitter/receiver device.

Using the input, a phlebotomist or operator may enter parameter and information relevant to the blood donation procedure. This information can include patient measurements such as height, weight, hematocrit, or characteristics such as gender, previous donation reactions, or any other relevant information. This information can also include process parameters. A phlebotomist or user may also enter donor specific parameters, including baseline parameters. These baseline parameters may have a minimum or maximum and/or acceptable range, which when measured parameters fall outside this range, the risk of donor reaction is higher. For example, if the temperature of the donor drops more than 2 degrees Celsius from the baseline parameter value, the donor may experience an adverse event. As another example, if the donor blood pressure drops 20% from the baseline parameter value, the donor may experience an adverse event.

More particularly, in carrying out any one of these blood donation or blood processing applications, the controller is configured and/or programmed to control the flow of a fluid from a patient or donor and adjusts the speed of the flow or the amount/flow rate of citrate being returned to the patient/donor. This may include controlling the adjustment mechanism to alter the flow rate through a flow line or the speed of the anticoagulant pump, which drives the return of blood components and citrate to the patient/donor. Any known method of adjusting rate of the flow into and out of the blood container may be used. Hence, while it may be described herein that a particular component of the blood donation system performs a particular function, it should be understood that that component is being controlled by the controller to perform that function.

Before, during, and after a procedure, the controller may receive readings from the donor parameter monitoring device and may have to adjust targets or process conditions based on these readings. For example, the controller may instruct one of the adjustment mechanism to control the flow rate of blood from a donor/patient and/or control the flow rate of the return of components including anticoagulant/citrate to the patient and/or the amount of citrate added to the return line to the donor.

Before a typical whole blood donation procedure, a user or phlebotomist may program the controller by the using the input to add donor specific characteristics and parameters. The donor is then phlebotomized using the vein access device 12. Whole blood is drawn into the system. During the blood draw, the donor parameter monitoring device occasionally and/or at selected intervals will measure the specific parameter of the donor. The parameters may also be done continuously, such as, for example, with an infrared camera. These measurements are compiled by the controller and analyzed in order to predict the probability of a donor reaction. The analysis may include comparing the measured parameters to baseline parameters entered before the blood draw. The measured parameters may also compare the measured parameters to standard parameters for people similar to the donor (i.e., height, weight, hematocrit level). If the controller calculates a donor reaction is likely to occur, then established adjustments to the features of the blood draw take place. For instance, an adjustment mechanism may move the blood container 16 or clamp the flow line 18 from the donor. The donor parameter monitoring device continues to measure the selected parameter and the controller continues to analyze, calculate the probability of a reaction, and adjust at least one feature of the blood draw as necessary until the blood draw is complete.

The current disclosure also includes possible blood component donation and component return methods, by the system 100 shown in FIG. 3. In typical component donation, such as apheresis systems, whole blood is drawn from a donor and combined with an amount of anticoagulant to retard coagulation during further processing. The anticoagulated blood is then processed through the system to obtain the needed separation. The blood components that are not stored for later use or removed for therapeutic purposes are typically returned to the donor or patient. Returned with the components will be at least a portion of the anticoagulant, commonly a citrate, which can also affect the donor. Thus, controlling the anticoagulant or citrate amount by one or more pumps can be an additional adjustment mechanism in systems with a blood component returned to donor.

Calculating and setting the anticoagulant or citrate rate or amount can be done before initiating a donation procedure. The amount or rate of citrate or anticoagulant returned to the donor can be calculated a number of ways. If there are separate anticoagulant and whole blood pumps, they may operate at a fixed ratio of whole blood flow rate to anticoagulant flow rate. The anticoagulant flow rate AC may also be a calculated rate at which the donor or patient can metabolize the anticoagulant or a component thereof. The citrate or anticoagulant rate/amount may also be adjusted depending on the donor's or patient's citrate tolerance. For donors known to be particularly sensitive to citrate (i.e., those requiring a lower CIR), the anticoagulant flow rate AC may be decreased to an appropriate level, with the resultant maximum whole blood draw rate also reduced. Similarly, for donors known to tolerate a higher CIR (e.g., a CIR approximately equal to 2 mg Citrate/kg Donor-weight/min), the anticoagulant flow rate AC may be increased to a higher acceptable level, with the resultant maximum whole blood draw rate also increased. Another factor in selecting an appropriate anticoagulant flow rate AC may be the nature of the blood donation procedure.

In blood donation system 100, the citrate/anticoagulant rate or amount can be entered or set by an operator along with other donor specific characteristics or parameters. Afterward, the donor is phlebotomized using the vein access device 112. The controller 130 operates the whole blood pump 128A (if provided) to draw whole blood from the donor into the system 100 at the draw flow rate WB. If a whole blood pump 128A is not provided, then the controller 130 directs the operation of one or more of the other pumps such that blood is drawn from the donor into the system at the draw flow rate WB. The controller 130 also operates the anticoagulant pump 128c (if provided) to pump anticoagulant from the anticoagulant source 114 into the system 110 at the appropriate anticoagulant flow rate AC. If an anticoagulant pump 128c is not provided, then the controller 130 directs the operation of one or more of the other pumps such that anticoagulant is added from the anticoagulant source 114 into the blood in the system 100 at the anticoagulant flow rate AC. The anticoagulated blood passes into the blood container 116 of a blood processing assembly which processes the blood. At least a portion (and potentially all in some procedures) of the processed anticoagulated blood is passed out of the blood container 116, through the return flow line 122 and returned to the donor via the vein access device 12. During the blood donation procedure, the donor parameter monitoring device occasionally and/or at selected intervals will measure the specific parameter of the donor. These measurements are compiled by the controller and analyzed in order to predict the probability of a donor reaction. The analysis may include comparing the measured parameters to baseline parameters entered before the blood draw. The measured parameters may also compare the measured parameters to standard parameters for people similar to the donor (i.e., height, weight, hematocrit level). If the controller calculates a donor reaction is likely to occur, then established or otherwise selected adjustments to the features of the blood draw take place. For instance, an adjustment mechanism may move the blood container 116 or clamp the flow line 118 from the donor. As discussed above, the AC may also be adjusted if the analyzed parameter indicates a probability of a citrate reaction. The donor parameter monitoring device continues to measure the selected parameter and the controller continues to analyze, calculate the probability of a reaction, and adjust at least one feature of the blood draw as necessary until the blood draw is complete.

Thus, improved blood donation systems and methods have been provided. The system and method should provide for the ability to anticipate a donor reaction and prevent or minimize the reaction.

### Aspects

Aspect 1. A method for reducing donor reactions during a blood donation using an automated blood draw device, the device including a controller, the method comprising: (a) initiating a blood draw; (b) monitoring at least one donor parameter during the blood draw; (c) analyzing the at least one donor parameter; (d) predicting the probability of a reaction in a donor; (e) adjusting at least one feature of the blood draw so that a donor reaction can be minimized or prevented if a reaction is determined to be probable; and (f) performing steps b-e until the blood draw is complete.

Aspect 2. The method of Aspect 1, wherein at least one baseline donor parameter is inputted into the blood draw device.

Aspect 3. The method of Aspect 2, wherein the at least one baseline donor parameter is a range that includes minimum and maximum values.

Aspect 4. The method of Aspect 2 or 3, wherein analyzing at least one parameter includes comparing the measured at least one parameter to the at least one baseline donor parameter.

Aspect 5. The method of Aspect 3, wherein predicting the probability of a reaction includes determining if the at least one parameter is outside of the at least one baseline donor parameter range.

Aspect 6. The method of any of the preceding Aspects, wherein adjusting at least one feature of the blood draw includes modifying the position of a blood collection container.

Aspect 7. The method of any of the preceding Aspects, wherein adjusting at least one feature of the blood draw includes tightening a clamp on a flow line of the blood draw device.

Aspect 8. The method of any of the preceding Aspects, wherein adjusting at least one feature of the blood draw includes decreasing the amount of citrate returned to the donor.

Aspect 9. The method of any of the preceding Aspects, wherein the at least one donor parameter includes at least one of pulse, temperature, blood pressure, heart rate, sweat, facial expression, and nerve conduction.

Aspect 10. The method of any of the preceding Aspects, wherein the monitoring at least one patient parameter includes using at least one of the following parameter sensors.

Aspect 11. A system for performing a blood draw procedure comprising: (i) a vein access device; (ii) a fluid circuit comprising tubing connected to a vein access device and at least one blood collection container for collecting blood and/or blood components; (iii) at least one donor parameter monitoring device; (iv) at least one adjusting mechanism for adjusting at least one feature of a blood draw; (v) a programmable user interface; (vi) a controller configured to communicate with the donor parameter monitoring device, the at least one adjusting mechanism, and the user interface; wherein the controller is configured to: (a) initiate a blood draw; (b) monitor at least one donor parameter during the blood draw; (c) analyze at least one donor parameter; (d) predict the probability of a reaction in a donor; (e) adjust at least one feature of the blood draw so that a donor reaction can be minimized or prevented if a reaction is determined to be probable; and (f) perform steps b-e until the blood draw is complete.

Aspect 12. The system of Aspect 11, wherein at least one baseline donor parameter is inputted into the blood draw device.

Aspect 13. The system of Aspect 12, wherein the at least one baseline donor parameter range includes minimum and maximum values.

Aspect 14. The system of Aspect 12 or 13, wherein analyze at least one parameter includes comparing the measured at least one parameter to the at least one baseline donor parameter.

Aspect 15. The system of Aspect 13, wherein predict the probability of a reaction includes determining if the at least one parameter is outside of the at least one baseline donor parameter range.

Aspect 16. The system of any of Aspects 11-15, wherein the at least one adjustment mechanism modifies the position of a blood collection container.

Aspect 17. The system of any of Aspects 11-16, wherein the at least one adjustment mechanism includes a clamp on a flow line of the blood draw device.

Aspect 18. The system of any of Aspects 11-17, wherein the at least one adjustment mechanism includes a pump on an anticoagulant line.

Aspect 19. The system of any of Aspects 11-18, wherein the at least one donor parameter includes at least one of pulse, temperature, blood pressure, heart rate, sweat, facial expression, and nerve conduction.

Aspect 20. The system of any of Aspects 11-19, wherein the at least one donor parameter monitoring device includes at least one of a blood pressure cuff, a pulse oximeter, an electrocardiogram, and a thermometer.

1. A method for reducing donor reactions during a blood donation using an automated blood draw device, the device including a controller, the method comprising:
a. initiating a blood draw;
b. monitoring at least one donor parameter during the blood draw;
c. analyzing the at least one donor parameter;
d. predicting the probability of a reaction in a donor;
e. adjusting at least one feature of the blood draw so that a donor reaction can be minimized or prevented if a reaction is determined to be probable; and
f. performing steps b-e until the blood draw is complete.

2. The method of claim 1, wherein at least one baseline donor parameter is inputted into the blood draw device.

3. The method of claim 2, wherein the at least one baseline donor parameter is a range that includes minimum and maximum values.

4. The method of claim 2, wherein analyzing at least one parameter includes comparing the measured at least one parameter to the at least one baseline donor parameter.

5. The method of claim 3, wherein predicting the probability of a reaction includes determining if the at least one parameter is outside of the at least one baseline donor parameter range.

6. The method of claim 1, wherein adjusting at least one feature of the blood draw includes modifying the position of a blood collection container.

7. The method of claim 1, wherein adjusting at least one feature of the blood draw includes tightening a clamp on a flow line of the blood draw device.

8. The method of claim 1, wherein adjusting at least one feature of the blood draw includes decreasing the amount of citrate returned to the donor.

9. The method of claim 1, wherein the at least one donor parameter includes at least one of pulse, temperature, blood pressure, heart rate, sweat, facial expression, and nerve conduction.

10. The method of claim 1, wherein the monitoring at least one patient parameter includes using at least one of the following parameter sensors.

11. A system for performing a blood draw procedure comprising:
i. a vein access device;
ii. a fluid circuit comprising tubing connected to a vein access device and at least one blood collection container for collecting blood and/or blood components;
iii. at least one donor parameter monitoring device;
iv. at least one adjusting mechanism for adjusting at least one feature of a blood draw;
v. a programmable user interface;
vi. a controller configured to communicate with the donor parameter monitoring device, the at least one adjusting mechanism, and the user interface; wherein the controller is configured to:
a. initiate a blood draw;
b. monitor at least one donor parameter during the blood draw;
c. analyze at least one donor parameter;
d. predict the probability of a reaction in a donor;
e. adjust at least one feature of the blood draw so that a donor reaction can be minimized or prevented if a reaction is determined to be probable; and
f. perform steps b-e until the blood draw is complete.

12. The system of claim 11, wherein at least one baseline donor parameter is inputted into the blood draw device.

13. The system of claim 12, wherein the at least one baseline donor parameter is a range includes minimum and maximum values.

14. The system of claim 12, wherein analyze at least one parameter includes comparing the measured at least one parameter to the at least one baseline donor parameter.

15. The system of claim 13, wherein predict the probability of a reaction includes determining if the at least one parameter is outside of the at least one baseline donor parameter range.

16. The system of claim 11, wherein the at least one adjustment mechanism modifies the position of a blood collection container.

17. The system of claim 11, wherein the at least one adjustment mechanism includes a clamp on a flow line of the blood draw device.

18. The system of claim 11, wherein the at least one adjustment mechanism includes a pump on an anticoagulant line.

19. The system of claim 11, wherein the at least one donor parameter includes at least one of pulse, temperature, blood pressure, heart rate, sweat, facial expression, and nerve conduction.

20. The system of claim 11, wherein the at least one donor parameter monitoring device includes at least one of a blood pressure cuff, a pulse oximeter, an electrocardiogram, and a thermometer.
